(19) United States
(12) Patent Application Publication
(10) Pub. No.: US 2024/0115588 A1
(43) Pub. Date: Apr. 11, 2024

(54) USE OF PROTEASOME INHIBITORS IN THE TREATMENT OF CORONAVIRUS INFECTIONS

(71) Applicants: THE BOARD OF SUPERVISORS OF LOUISIANA STATE UNIVERSITY AND AGRICULTURAL AND MECHANICAL COLLEGE, Baton Rouge, LA (US); Skymount Medical US lnc., New Orleans, LA (US)

(72) Inventors: Adam Bess, Baton Rouge, LA (US); Frej Knut Gosta BERGLIND, Baton Rouge, LA (US); Supratik MUKHOPADHYAY, Baton Rouge (JP); Kishor M. WASAN, Saskatoon, Saskatchewan (CA); Chris GALLIANO, New Orleans, LA (US); Michal BRYLINSKI, Baton Rouge, LA (US); Stephania CORMIER, Baton Rouge, LA (US); Allan ADER, New Orleans, LA (US); Nicholas GRIGGS, New Orleans, LA (US); Janet GOULD, New Orleans, LA (US); Tiffany CHO, New Orleans, LA (US); Julia ABRAMOV, New Orleans, LA (US); Peter HNIK, New Orleans, LA (US)

(73) Assignees: Board of Supervisors of Louisiana state University and Agricultural and Mechanical College, Baton Rouge, LA (US); Skymount Medical US lnc., New Orleans, LA (US)

(21) Appl. No.: 18/275,365

(22) PCT Filed: Feb. 1, 2022
(86) PCT No.: PCT/US2022/014726

§ 371 Date: Aug. 1, 2023

(30) Foreign Application Priority Data
Feb. 1, 2021 (CA) 3107602

(51) Int. Cl. A61K 31/69; A61K 9/00; A61K 38/07; A61P 31/14

(52) U.S. Cl. CPC A61K 31/69; A61K 9/0053; A61K 38/07; A61P 31/14

(57) ABSTRACT

Oral formulations comprising one or more proteasome inhibitors for use as a treatment of coronavirus infection in a subject. Also disclosed are the use of such for the treatment or prevention of SARS-CoV-2 infection in humans.

### TECHNICAL FIELD

The present disclosure is directed to the treatment of coronavirus diseases, including the use of oral formulations of proteasome inhibitors for the treatment of COVID-19 infections in humans.

### BACKGROUND

The appearance of a novel coronavirus, referred to as SARS-CoV-2, on the world stage has affected substantially every population in the world. This virus has afflicted millions of individuals and caused a disease, referred to as COVID-19. COVID-19 can develop into a significant health risk and result in death, which has placed a high strain on healthcare resources and society in general.

SARS-CoV-2 is a single-strand, positive-sense ribonucleic acid (RNA) virus with a similar receptor-binding domain structure to that of SARS-CoV and MERS-CoV. SARS-CoV-2 is transmitted between individuals via airborne droplets accessing nasal mucosa. Within the nasal mucosa SARS-CoV-2 can rapidly reproduce and be shed in nasal secretions (sputum). Sputum can be transmitted to other individuals via airborne droplets, thus repeating the transmission cycle. The SARS-CoV-2 virus can spread between individuals before the onset of symptoms, during the symptomatic period and even after recovery.

The clinical spectrum of the infection is wide, ranging from mild signs of an upper respiratory tract infection to severe pneumonia, multi-organ failure and death. At the onset, SARS-CoV-2 primarily attacks the respiratory system, as it represents the main point of entry into the host, but SARS-CoV-2 also can affect multiple organs of an infected individual. The severity of COVID-19 is typically associated with comorbidities such as, but not limited to: hypertension, diabetes, obesity, and/or advanced age that can exacerbate the consequences of COVID-19.

A number of vaccines for SARS-CoV-2 have become available and form an important part of health official's recommendations to ease the COVID-19 public health crisis and to manage the stress COVID-19 has placed on health care workers and hospitals. However, the SARS-CoV-2 virus has already demonstrated the ability to mutate into various different variants, some of which are more transmissible than other variants and some of which are proving successful at avoiding the protections afforded by current vaccines.

There exists a need for a therapy that is capable of mitigating the impact of the SARS-CoV-2 virus in such a manner that it slows down, prevents and/or treats the physiological impact on an infected individual.

### SUMMARY

The embodiments of the present disclosure provide one or more therapies for ameliorating and/or inhibiting some or substantially all of the risks, symptoms and development of severe disease in a subject infected with a coronavirus. In some embodiments of the present disclosure, the coronavirus is SARS-CoV-2.

Some embodiments of the present disclosure relate to a use of one or more proteasome inhibitors for ameliorating and/or inhibiting some or substantially all of the risks, symptoms and development of severe disease caused by a coronavirus infection.

Some embodiments of the present disclosure relate to a use of an oral formulation comprising one or more proteasome inhibitors for preventing a SARS-CoV-2 infection in a subject.

Some embodiments of the present disclosure relate to a use of an oral formulation comprising one or more proteasome inhibitors for treating a SARS-CoV-2 infection in a subject.

Some embodiments of the present disclosure relate to a use of an oral formulation comprising one or more proteasome inhibitors for preventing replication of SARS-CoV-2 virus in a subject.

Some embodiments of the present disclosure relate to a method of treating an individual exposed to or infected with a coronavirus. The method comprises the steps of providing a therapeutically effective amount of one or more proteasome inhibitors; and, administering the therapeutically effective amount one or more proteasome inhibitors to said individual to ameliorate and/or inhibit some or substantially all of the risks, symptoms and development of severe disease in a subject exposed to or infected with a coronavirus.

Some embodiments of the present disclosure relate to a method of making an agent/target cell complex, the method comprising a step of administering a therapeutically effective amount of the agent to a subject, wherein the agent/target cell complex inhibits, delays or prevents virion particles from entering a target cell that forms part of the complex, fusing with the target cell and/or replicating within the target cell. In some embodiments of the present disclosure, the agent comprises one or more proteasome inhibitors.

Some embodiments of the present disclosure relate to a method of making an agent/target virion complex, the method comprising a step of administering a therapeutically effective amount of the agent to a subject, wherein the agent/target virion complex inhibits the agent/target virion complex from entering a subject's cell, fusing with a subject's cell and/or replicating within a subject's cell. In some embodiments of the present disclosure, the agent comprises one or more proteasome inhibitors.

In the embodiments of the present disclosure, the proteasome inhibitors include bortezomib, ixazomib, carfilzomib or combinations thereof. Ixazomib is currently available for oral administration and may have improved activity over bortezomib and/or carfilzomib. Bortezomib and carfilzomib are both typically administered by injection or intravenously. Bortezomib is a slowly reversible inhibitor (dissociation half-life=110 minutes) of the β1 caspase-like subunit and β2 trypsin-like subunit, with preference to the β5 chymotrypsin-like subunit of the 20S proteolytic site of the proteasome. Carfilzomib is an irreversible inhibitor with a high specificity for the β5 chymotrypsin-like subunit of the proteasome. Ixazomib and bortezomib are mechanistically similar, where they both have a greater affinity for the β5 chymotrypsin-like subunit of the proteasome; however, ixazomib has a dissociation half-life of 18 minutes, which is believed to contribute to its superior tissue penetration. Proteasomes are highly concentrated in blood cells and bortezomib is known to retain a longer exposure in circulation where it exerts most of its inhibitory activity. At higher concentrations, ixazomib can also inhibit other proteolytic sites (e.g., β1, β2).

The initial in vitro studies described herein have shown promise in treating both kidney cells and lung cells with one or more proteasome inhibitors, when such cells have been exposed to SARS-CoV-2 virus. The EC50 values have been in the low nM range indicating specificity and potency against SARS-CoV2.

Current formulations of bortezomib are limited to IV administration or subcutaneous injection. The advantages of such include a 100% bioavailability and wide distribution to peripheral tissues. After IV administration, the time to peak plasma levels is approximately 5 minutes. In vitro binding of IV administered bortezomib to human plasma protein averaged 83%.

Cyclins and CDK inhibitors regulate the activity of CDKs, and, in turn, the proteasome regulates these proteins. As well, it has been shown that the combination of citreoviridin and the 26S proteasome inhibitor bortezomib could improve the anticancer activity by enhancing ER stress, by ameliorating citreoviridin-caused cyclin D3 compensation, and by contributing to CDK1 [cyclin-dependent kinase 1] deactivation and PCNA downregulation. In light of this, the inventors postulated that bortezomib may be effective against SARS-CoV-2 because it inhibits CDK activity.

In vitro data show that bortezomib treated SARS-CoV-2-infected Vero E6 cells demonstrated inhibited virus-induced cytopathic effects at a concentration of 0.05 μM. However, cytotoxicity was observed at 0.002 μM. Cytotoxicity and inhibition of the virus-induced cytopathic effects were observed at doses >30 μM with a shorter treatment schedule. Further data indicates effectiveness in infected kidney and lung cells. However, an oral dosage formulation of bortezomib may address these drawbacks.

Given the above, the utility of this bortezomib may be limited by its route of administration, narrow therapeutic index, the frequency and/or severity of clinical adverse effects that may be observed after chronic exposure (e.g., respiratory distress, cardiovascular disturbances), including effects on the developing fetus and its potential to impair fertility.

Without being bound by any particular theory, the proteasome inhibitor bortezomib may be useful in treating and/or preventing SARS-CoV-2 infection because of it inhibits CDK activity. Bortezomib is known to target proteasome subunit beta type-1 (0.5 nM), type-2 (N/A), type-5 (0.5 nM), type-7 (7 nM); type-8 (17 nM); 20S proteasome chymotrypsin-like (1.90 nM); proteasome subunit beta-type1/beta type-5 (4 nM); proteasome subunit beta type-7 (7 nM); 26S proteasome (8.10 nM); proteasome subunit beta type-8 (17 nM); proteasome component C5 (30 nM); proteasome component C5 (130 nM); proteasome; macropain subunit (440 nM); and cathepsin G (520 nM). Bortezomib is also known to target: cathepsin A (9200 nM), Cathepsin B (>3000 nM), and Cathepsin G (520 nM); chymase (Mast cell protease 1) (1190 nM). Bortezomib is also known to target multidrug resistance-associated protein 4 (ABCC4) (133 μM), Canalicular multispecific organic anion transporter 1 (ABCC2) (133 μM), Canalicular multispecific organic anion transporter 2 (ABCC3) (133 μM), Bile salt export pump (ABCB11) (133 μM).

Without being bound by any particular theory, the proteasome inhibitor ixazomib may be useful in treating and/or preventing SARS-CoV-2 infection because of its mechanism of action as it is known to target: proteasome subunit beta type-1 (7.7 nM), type-2 (N/A), and type-5 (7.7 nM).

Without being bound by any particular theory, the proteasome inhibitor carfilzomib may be useful in treating and/or preventing SARS-CoV-2 infection because of its mechanism of action as it is known to target: proteasome subunit beta type-5 (9.6 nM), type-7 (8.6 nM), type-8 (N/A); Cathepsin A (>30 μM), Cathepsin B (11 μM), and Cathepsin G (>30 μM).

### BRIEF DESCRIPTION OF THE FIGURES

The features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 is a graphical representation of the concentration-response curves for remdesevir from the second testing series; and

FIG. 2 is a graphical representation of the concentration-response curves for bortezomib from the second testing series.

FIG. 3 is a graphical representation of the concentration-response curves for remdesevir in Calu3 cells.

FIG. 4 is a graphical representation of the concentration-response curves for carfilzomib in Calu3 cells.

### DETAILED DESCRIPTION

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “activity” is used interchangeably with the term “functionality” and both terms refer to the physiologic action of a biomolecule.

As used herein, the terms “agent” and “therapeutic agent” refer to a substance that, when administered to a subject, causes one or more chemical reactions and/or one or more physical reactions and/or or one or more physiological reactions and/or one or more pharmacological reactions and/or one or more immunological reactions in the subject.

As used herein, the term “ameliorate” refers to improve and/or to make better and/or to make more satisfactory.

As used herein, the term “cell” refers to a single cell as well as a plurality of cells or a population of the same cell type or different cell types. Administering an agent to a cell includes in vivo, in vitro and ex vivo administrations and/or combinations thereof.

As used herein, the term “complex” refers to an association, either direct or indirect, between one or more particles of an agent and one or more target cells or target virions. This association results in a change in the metabolism or functionality of the target cells or target virions. As used herein, the phrase “change in metabolism” refers to an increase or a decrease in the one or more of the targets' production of one or more proteins, and/or any post-translational modifications of one or more proteins. As used herein, the phrase “change in functionality” refers to a difference in physiological function of one or more aspects of the target within an agent/target complex as compared to a target that is not part of such a complex.

As used herein, the terms “dysregulation” and “dysregulated” refer to situations or conditions wherein homeostatic control systems have been disturbed and/or compromised so that one or more metabolic, physiologic and/or biochemical systems within a subject operate partially or entirely without said homeostatic control systems.

As used herein, the term “excipient” refers to any substance, not itself an agent, which may be used in a composition for delivery of one or more agents, and the like to a subject or alternatively combined with . . . one or more carriers and the like (e.g., to create a pharmaceutical composition) to improve its handling or storage properties or to permit or facilitate formation of a dose unit of the composition (e.g., formation of a topical hydrogel which may then be optionally incorporated into a transdermal patch). Excipients include, by way of illustration and not limitation, binders, disintegrants, taste enhancers, solvents, thickening or gelling agents (and any neutralizing agents, if necessary), penetration enhancers, solubilizing agents, wetting agents, antioxidants, lubricants, emollients, substances added to mask or counteract a disagreeable odor, fragrances or taste, substances added to improve appearance or texture of the composition and substances used to form the pharmaceutical compositions. Any such excipients can be used in any dosage forms according to the present disclosure. The foregoing classes of excipients are not meant to be exhaustive but merely illustrative.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%, 100%, or any amount of reduction in between the specifically recited percentages, as compared to native or control levels.

As used herein, the term “medicament” refers to a medicine and/or pharmaceutical composition that comprises an agent and that can promote recovery from a disease, disorder or symptom thereof and/or that can prevent a disease, disorder or symptom thereof and/or that can inhibit the progression of a disease, disorder, or symptom thereof.

As used herein, the term “pharmaceutical composition” means any composition comprising, but not necessarily limited to, one or more agents to be administered a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the one or more agents. Some examples of pharmaceutically acceptable carriers and their formulations are described in Remington (1995, The Science and Practice of Pharmacy (19th ed.) ed. A. R. Gennaro, Mack Publishing Company, Easton, PA), the disclosure of which is incorporated herein by reference. Typically, an appropriate amount of a pharmaceutically acceptable carrier is used in the formulation to render said formulation isotonic. Examples of suitable pharmaceutically acceptable carriers include, but are not limited to: saline solutions, glycerol solutions, ethanol, N-(1(2, 3-dioleyloxy)propyl)-N,N,N-trimethylammonium chloride (DOTMA), dioleolphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation suits the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevent”, “prevention of” and “preventing” refer to avoiding the onset or progression of a disease, disorder, or a symptom thereof.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types, ex vivo preparations, and/or a sample of biological materials such as tissue and/or biological fluids.

As used herein, the term “target cell” refers to one or more cell types within a subject that can interact with a coronavirus by the virus fusing with the outer membrane of the one or more cell types, entering into the cell and/or replicating therein. Without being bound to any particular theory, target cells of a subject can include any cells within a subject that express the receptors and/or co-factors required for viral interaction. Examples of these types of cells include, but are not limited to: epithelial cells of the upper airways and conducting airways (ciliated and non-ciliated); alveolar epithelial cells (both type 1 and 2); epithelial cells and neurons of the olfactory system; neurons of the central or peripheral nervous system; epithelial cells, enteroctytes and gland cells of the gastrointestinal tract; cells of the blood, including immune effector cells; cardiovascular cells, and renal cells.

As used herein, the term “target virion” and “virion” refer to one or more viral particles of coronavirus that have the capacity to cause a viral infection within a target cell. In some embodiments of the present disclosure, the viral particles are of one or more variants of SARS-CoV-2.

As used herein, the term “therapeutically effective amount” refers to the amount of the agent used that is of sufficient quantity to ameliorate, prevent, treat and/or inhibit one or more of a disease, disorder or a symptom thereof. The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or the effect may be therapeutic in providing a partial or complete amelioration or inhibition of a disease, disorder, or symptom thereof. Additionally, the term “treatment” refers to any treatment of a disease, disorder, or symptom thereof in a subject and includes: (a) preventing the disease from occurring in a subject which may be predisposed to the disease but has not yet been diagnosed as having it; (b) inhibiting the disease, i.e., arresting its development; and, (c) ameliorating the disease.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more additional active ingredients, or combinations thereof. The amount of agent within each unit is a therapeutically effective amount.

In embodiments of the present disclosure, the pharmaceutical compositions disclosed herein comprise one or more agents as described above in a total amount by weight of the composition of about 0.1% to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%. about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95% or more.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The embodiments of the present disclosure relate to use of one or more proteasome inhibitors for preventing, reducing and/or treating infection with a SARS-CoV-2 virus in a subject. In the embodiments of the present disclosure, the proteasome inhibitors include bortezomib, ixazomib, carfilzomib or combinations thereof.

Bortezomib is indicated for the treatment of patients with multiple myeloma and mantle cell lymphoma. It is given as a combination treatment and can be administered subcutaneously (SC) or intravenously (IV). The recommended starting dose for injection is 1.3 mg/m2 (corresponding to 2.3 mg/day, if administered daily, based on an average body surface area of 1.8 m2).

Bortezomib is currently used as a potent, selective, and reversible inhibitor of the 26S proteasome, a large protein complex that degrades ubiquitinated proteins in mammalian cells. The ubiquitin-proteasome pathway is important in regulating the intracellular concentration of specific proteins to maintain homeostasis within cells. Although in cancer cells, blocking this pathway can affect multiple cell signaling cascades that lead to the inhibition of NF-κB activation and cell cycle progression, and the initiation of apoptosis. Proteasome inhibition may also lead to the accumulation of cyclin-dependent kinase (CDK) inhibitors, such as p27.

In an in vitro study, bortezomib was shown to inhibit the virus-induced cytopathic effects (CPE) at 0.05 μM in Vero E6 cells infected with SARS-CoV-2; however, unfavorable cytotoxicity occurred at 0.002 μM. With a shorter drug treatment time, bortezomib could not completely prevent CPE at doses >30 μM as effectively as chloroquine (which was achieved at 15 μM).

Additionally, bortezomib has been identified as a drug with potential activity against SARS-CoV-2 based on analytical and computational approaches. The open reading frame 10 (ORF10) viral protein has been identified as a key protein responsible for the highly contagious nature of SARS-CoV-2 viral particles and has been reported to interact with the E3 ligase complex, which plays a role in targeting cellular proteins for ubiquitination by the 26S proteasome. This suggests that ORF10 may bind to the proteasome complex and exploit it for the ubiquitination and degradation of restriction factors and other essential cellular proteins. Furthermore, bortezomib was categorized as a cytotoxic drug in Vero E6 cells by an algorithmic prediction study using artificial intelligence, network diffusion, and network proximity to rank a number of drugs for their expected efficacy against SARS-CoV-2.

It has been shown that SARS-CoV-2 virus increases the phosphorylation and activation of CDKs, which leads to an increased supply of essential nucleotides, DNA repair, and replication proteins that are essential for viral replication. Without being bound by any particular theories, CDK inhibitors are potential therapies for the treatment of SARS-CoV-2 viral infection, the inhibition of the proteasome leads to the accumulation of CDK inhibitors and the downregulation of NF-κB-mediated inflammation. As such, using a proteasome inhibitor may be advantageous in the context of mitigating SARS-CoV-2 infection and decreasing the severity of COVID-19.

The unfolded protein response (UPR) is a signaling pathway activated by the accumulation of misfolded proteins within the endoplasmic reticulum (ER) of the cell. Activation of this pathway leads to the increased production of molecular chaperones, suppression of protein translation, and the accelerated degradation of misfolded proteins. The SARS-CoV-2 virus exploits the endogenous transcriptional machinery for the generation of viral proteins, and as a result of rapid viral replication, unfolded viral polypeptides often accumulate in the ER. When the system is overburdened by viral proteins, the production of endogenous proteins is suppressed, leading to cell death. Proteasome inhibitors initiate the UPR through the induction of the protein kinase R-like endoplasmic reticulum kinase (PERK) and activating transcription factor 4 (ATF4) for the removal of unfolded or aggregated proteins. Without being bound by any particular theory, one or more proteasome inhibitors may increase the capacity of the UPR to uphold normal cell integrity and function. Pharmacological chaperone therapy to treat COVID-19 patients has been considered; however, prolonged UPR activation and severe ER stress may be associated with other disease states (e.g., Alzheimer's disease, pulmonary fibrosis).

In light of the current state of the art, there exists a need for therapeutic compounds capable of reducing and/or ameliorating the physiological impact of the SARS-CoV-2 virus on an infected individual. The present disclosure meets this need by providing compositions and methods for the treatment of coronavirus infections, including SARS-CoV-2 infection, and related diseases and disorders.

In some embodiments of the present disclosure, the coronavirus is SARS-CoV-2 and use of one or more proteasome inhibitors may inhibit replication of the SARS-CoV-2 virus. In some embodiments of the present disclosure, the subject may be infected with or at risk of infection by SARS-CoV-2, or has been diagnosed with or suspected to have COVID-19. In some embodiments of the present disclosure, the subject is a human. In some embodiments of the present disclosure, the proteasome inhibitor is provided orally or is formulated for oral administration. In some embodiments of the present disclosure, the subject is provided an oral formulation of bortezomib, including but not limited to those oral formulations described in any of Tables 4-8, herein below.

Protein-Protein Binding

A thorough assessment of the potential of small therapeutics to bind with COVID-19 virus particles was carried out. Using three different mechanism potential binding sites for small molecules, the likelihood of protein-protein binding was determined. Using a template of the crystal structure of an essential SARS-CoV-2 protease, the functional centers of the protease inhibitor-binding pocket were identified.

Antiviral peptides known to inhibit the SARS virus were used as targets. By creating a fingerprint (embedding) of these antiviral peptides (AVPs) one then compared them to similarly generated fingerprints (embedding) of individual drugs to identify the ones most closely related.

The AVPs used targeted three specific mechanisms: Entry, Fusion, and Replication. The most effective peptides were specifically filtered out and used those to create three separate networks based on each peptide's known mechanism of action. This allowed the identification of drugs with certain specificities based on mechanism.

The three mechanisms are relevant for the following reasons. Entry is extremely important because inhibiting viral entry into the cell would reduce the amount of virus that acts on the cell. Likewise, inhibition of replication is important for reducing the amount of viral load generated and spread to other cells after a cell has been infected. Finally, fusion though technically least relevant is worth noting because not all viral entry happens through the standard mechanism. The virus is capable of fusing directly with the membrane of the cell for infection. Though this happens at about 1/10th the rate of the standard entry mechanism, it is still a mechanism which was desirable to use as a focus to attempt to inhibit.

The fingerprints of these specific peptides were created by using the human proteome and a large graph of the proteins involved in all the processes therein. By then comparing these fingerprints to the drug fingerprints, the identification of drugs with a similar (antiviral) effect on the human proteome as the AVPs was carried out.

First Binding Mechanism

A number of therapeutic compounds where studied to determine their propensity to bind to SARS-CoV-2 viral particles according to a first binding mechanism. The interactions where further evaluated by assessing the likelihood the therapeutic compounds would impact the entry of COVID-19 into mammalian cells; the fusion of SARS-CoV-2 viral with mammalian cells; and ultimately the replication of the COVID-19 infected cells. Table 1 summarizes the data obtained in this first round of modeling data analysis.

TABLE 1
Results of Protein-Protein modeling data which mimics a
first mechanism of interaction between COVID-19 and each
one of the proposed therapeutic treatment molecules

| | | AI Network MLP (>0.25 = favorable scores) | | | | |
|---|---|---|---|---|---|---|
| | AI total | | | | | Entry, Fusion, |
| | score | Corona | Entry | Fusion | Replication | and/or Replication? |
| Bortezomib | 0.06 | 0.2699 | 0.1679 | 0.0715 | 0.0089 | — |
| Ixazomib | 0.94 | 0.843 | 0.87 | 0.1155 | 0.8225 | Entry & Replication |

According to the data collected in the study of the first binding mechanism, a majority of the compounds (those having a measured score of greater than 0.25) analyzed demonstrated a propensity to bind to SARS-CoV-2 viral particles.

Second Binding Mechanism

The same therapeutic compounds were subsequently studied to determine their propensity to bind to SARS-CoV-2 viral particles according to a second binding mechanism. The interactions where also further evaluated by assessing the likelihood the therapeutic compounds would impact the entry of COVID-19 into mammalian cells; the fusion of SARS-CoV-2 viral particles with mammalian cells; and ultimately the replication of the COVID-19 infected cells. Table 2 summarizes the data obtained in this second round of modeling data analysis.

TABLE 2
Results of Protein-Protein modeling data which mimics a
second mechanism of interaction between COVID-19 and each
one of the proposed therapeutic treatment molecules

| | AI Network Snet (higher is better) | | |
|---|---|---|---|
| | (<0.5 = unfavorable scores) | | |
| | Entry | Fusion | Replication |
| Bortezomib | 0.6058 | 0.5762 | 0.6013 |
| Ixazomib | 0.7938 | 0.7259 | 0.8877 |

According to the data collected in the study of the second binding mechanism, all of the compounds (those having a measured score of greater than 0.5) analyzed demonstrated a propensity to bind to SARS-CoV-2 viral particles.

Third Binding Mechanism

The same therapeutic compounds were again subsequently studied to determine their propensity to bind to SARS-CoV-2 viral particles according to a third binding mechanism. The interactions where also further evaluated by assessing the likelihood the therapeutic compounds would impact the entry of COVID-19 into mammalian cells; the fusion of SARS-CoV-2 viral particles with mammalian cells; and ultimately the replication of the COVID-19 infected cells. Table 3 summarizes the data obtained in this third round of modeling data analysis.

TABLE 3
Results of Protein-Protein modeling data which mimics a
third mechanism of interaction between COVID-19 and each
one of the proposed therapeutic treatment molecules

| | AI Network Cos Sim (higher = better) | | |
|---|---|---|---|
| | Entry | Fusion | Replication |
| Bortezomib | 0.485832051 | 0.455458353 | 0.527880143 |
| Ixazomib | 0.651902935 | 0.45575779 | 0.737173868 |

In some embodiments of the present disclosure relate to a method of inhibiting replication of a coronavirus in a mammal in need thereof, comprising providing to the mammal an effective amount of one or more proteasome inhibitors. In some embodiments of the present disclosure, the coronavirus is SARS-CoV-2. In some embodiments of the present disclosure, the subject in need of treatment using one or more proteasome inhibitors is infected with SARS-CoV-2, or has been diagnosed with COVID-19.

In certain embodiments, the one or more proteasome inhibitors are provided to the subject in an oral dose formulation comprising,
(a) a therapeutically effective amount of the one or more proteasome inhibitors;
(b) one or more fatty acid glycerol esters; and
(c) one or more polyethylene oxide-containing phospholipids or one or more polyethylene oxide-containing fatty acid esters.

In one embodiment, the one or more proteasome inhibitors is present in the formulation in an amount from about 0.5 to about 10 mg. In one embodiment, the one or more proteasome inhibitors is present in the formulation in about 3.5 mg. In particular embodiments, the formulation comprises: (a) the one or more proteasome inhibitors; (b) one or more fatty acid glycerol esters; (c) one or more polyethylene oxide-containing fatty acid esters; and, optionally, (d) a tocopherol polyethylene glycol succinate.

In one embodiment, the fatty acid glycerol esters comprise from about 32 to about 52% by weight fatty acid monoglycerides. In one embodiment, the fatty acid glycerol esters comprise from about 30 to about 50% by weight fatty acid diglycerides. In one embodiment, the fatty acid glycerol esters comprise from about 5 to about 20% by weight fatty acid triglycerides. In one embodiment, the fatty acid glycerol esters comprise greater than about 60% by weight oleic acid mono-, di-, and triglycerides.

In one embodiment, the polyethylene oxide-containing phospholipids comprise a C8-C22 saturated fatty acid ester of a phosphatidyl ethanolamine polyethylene glycol salt. In one embodiment, the polyethylene oxide-containing phospholipids comprise a distearoylphosphatidyl ethanolamine polyethylene glycol salt. In one embodiment, the distearoylphosphatidyl ethanolamine polyethylene glycol salt is selected from the group consisting of a distearoylphosphatidyl ethanolamine polyethylene glycol 350 salt, a distearoylphosphatidyl ethanolamine polyethylene glycol 550 salt, a distearoylphosphatidyl ethanolamine polyethylene glycol 750 salt, a distearoylphosphatidyl ethanolamine polyethylene glycol 1000 salt, a distearoylphosphatidyl ethanolamine polyethylene glycol 2000 salt, and mixtures thereof. In one embodiment, the distearoylphosphatidyl ethanolamine polyethylene glycol salt is present in the formulation in an amount from 1 mM to about 30 mM based on the volume of the formulation. In one embodiment, the distearoylphosphatidyl ethanolamine polyethylene glycol salt is an ammonium salt or a sodium salt.

In one embodiment, the polyethylene oxide-containing fatty acid esters comprise a polyethylene oxide ester of a C8-C22 saturated fatty acid. In one embodiment, the polyethylene oxide-containing fatty acid esters comprise a polyethylene oxide ester of a C12-C18 saturated fatty acid. In one embodiment, the polyethylene oxide-containing fatty acid esters is selected from the group consisting of: lauric acid esters, palmitic acid esters, stearic acid esters, and mixtures thereof. In one embodiment, the polyethylene oxide-containing fatty acid esters comprise a polyethylene oxide having an average molecular weight of from about 750 to about 2000. In one embodiment, the ratio of the fatty acid glycerol esters to polyethylene oxide-containing fatty acid esters is from about 20:80 to about 80:20 v/v. In one embodiment, the ratio of the fatty acid glycerol esters to polyethylene oxide-containing fatty acid esters is about 60:40 v/v. In one embodiment, the formulation further comprises glycerol in an amount less than about 10% by weight. In one embodiment, the formulation is a self-emulsifying drug delivery system.

In certain embodiments, the formulation further comprises a tocopherol polyethylene glycol succinate. In particular embodiments, the tocopherol polyethylene glycol succinate is present in the formulation from about 0.1 to about 10 percent by volume based on the total volume of the formulation. Structurally, tocopherol polyethylene glycol succinates have a polyethylene glycol (PEG) covalently coupled to tocopherol (e.g., α.-tocopherol or vitamin E) through a succinate linker. Because PEG is a polymer, a variety of polymer molecular weights can be used to prepare the TPGS. In one embodiment, the TPGS is tocopherol polyethylene glycol succinate 1000, in which the average molecular weight of the PEG is 1000. One suitable tocopherol polyethylene glycol succinate is vitamin E TPGS commercially available from Eastman. In one embodiment, the tocopherol polyethylene glycol succinate is present in the formulation in about 5 percent by volume based on the total volume of the formulation. In one embodiment, the formulation further comprises glycerol in an amount less than about 10% by weight. In one embodiment, the formulation is a self-emulsifying drug delivery system.

Certain bortezomib formulations disclosed herein include one or more fatty acid glycerol esters, and typically, a mixture of fatty acid glycerol esters. The fatty acid glycerol esters useful in the formulations can be provided by commercially available sources. A representative source for the fatty acid glycerol esters is a mixture of mono-, di-, and triesters commercially available as PECEOL® (Gattefosse, Saint Priest Cedex, France), commonly referred to as “glyceryl oleate” or “glyceryl monooleate.” When PECEOL® is used as the source of fatty acid glycerol esters in the formulations, the fatty acid glycerol esters comprise from about 32 to about 52% by weight fatty acid monoglycerides, from about 30 to about 50% by weight fatty acid diglycerides, and from about 5 to about 20% by weight fatty acid triglycerides. The fatty acid glycerol esters comprise greater than about 60% by weight oleic acid (C18:1) mono-, di-, and triglycerides. Other fatty acid glycerol esters include esters of palmitic acid (C16) (less than about 12%), stearic acid (C18) (less than about 6%), linoleic acid (C18:2) (less than about 35%), linolenic aid (C18:3) (less than about 2%), arachidic acid (C20) (less than about 2%), and eicosenoic acid (C20:1) (less than about 2%). PECEOL® can also include free glycerol (typically about 1%). In one embodiment, the fatty acid glycerol esters comprise about 44% by weight fatty acid monoglycerides, about 45% by weight fatty acid diglycerides, and about 9% by weight fatty acid triglycerides, and the fatty acid glycerol esters comprise about 78% by weight oleic acid (C18:1) mono-, di-, and triglycerides. Other fatty acid glycerol esters include esters of palmitic acid (C16) (about 4%), stearic acid (C18) (about 2%), linoleic acid (C18:2) (about 12%), linolenic acid (C18:3) (less than 1%), arachidic acid (C20) (less than 1%), and eicosenoic acid (C20:1) (less than 1%).

As used herein, the term “polyethylene oxide-containing fatty acid ester” refers to a fatty acid ester that includes a polyethylene oxide group (i.e., polyethylene glycol group) covalently coupled to the fatty acid through an ester bond. Polyethylene oxide-containing fatty acid esters include mono- and di-fatty acid esters of polyethylene glycol. Suitable polyethylene oxide-containing fatty acid esters are derived from fatty acids including saturated and unsaturated fatty acids having from eight (8) to twenty-two (22) carbons atoms (i.e., a polyethylene oxide ester of a C8-C22 fatty acid). In certain embodiments, suitable polyethylene oxide-containing fatty acid esters are derived from fatty acids including saturated and unsaturated fatty acids having from twelve (12) to eighteen (18) carbons atoms (i.e., a polyethylene oxide ester of a C12-C18 fatty acid). Representative polyethylene oxide-containing fatty acid esters include saturated C8-C22 fatty acid esters. In certain embodiments, suitable polyethylene oxide-containing fatty acid esters include saturated C12-C18 fatty acids. The molecular weight of the polyethylene oxide group of the polyethylene oxide-containing fatty acid ester can be varied to optimize the solubility of the therapeutic agent (e.g., the one or more proteasome inhibitors) in the formulation. Representative average molecular weights for the polyethylene oxide groups can be from about 350 to about 2000. In one embodiment, the average molecular weight for the polyethylene oxide group is about 1500. In this embodiment, the one or more proteasome inhibitors formulations include one or more polyethylene oxide-containing fatty acid esters, and typically, a mixture of polyethylene oxide-containing fatty acid esters (mono- and di-fatty acid esters of polyethylene glycol). The polyethylene oxide-containing fatty acid esters useful in the formulations can be provided by commercially available sources. Representative polyethylene oxide-containing fatty acid esters (mixtures of mono- and diesters) are commercially available under the designation GELUCIRE® (Gattefosse, Saint Priest Cedex, France).

Suitable polyethylene oxide-containing fatty acid esters can be provided by GELUCIRE® 44/14, GELUCIRE® 50/13, and GELUCIRE® 53/10. The numerals in these designations refer to the melting point and hydrophilic/lipophilic balance (HLB) of these materials, respectively. GELUCIRE® 44/14, GELUCIRE® 50/13, and GELUCIRE® 53/10 are mixtures of (a) mono-, di-, and triesters of glycerol (glycerides) and (b) mono- and diesters of polyethylene glycol (macrogols). The GELUCIRE® can also include free polyethylene glycol (e.g., PEG 1500). Lauric acid (C12) is the predominant fatty acid component of the glycerides and polyethylene glycol esters in GELUCIRE® 44/14. GELUCIRE® 44/14 is referred to as a mixture of glyceryl dilaurate (lauric acid diester with glycerol) and PEG dilaurate (lauric acid diester with polyethylene glycol), and is commonly known as PEG-32 glyceryl laurate (Gattefosse) lauroyl macrogol-32 glycerides EP, or lauroyl polyoxylglycerides USP/NF. GELUCIRE® 44/14 is produced by the reaction of hydrogenated palm kernel oil with polyethylene glycol (average molecular weight 1500). GELUCIRE® 44/14 includes about 20% mono-, di- and, triglycerides, about 72% mono- and di-fatty acid esters of polyethylene glycol 1500, and about 8% polyethylene glycol 1500. GELUCIRE® 44/14 includes lauric acid (C12) esters (30 to 50%), myristic acid (C14) esters (5 to 25%), palmitic acid (C16) esters (4 to 25%), stearic acid (C18) esters (5 to 35%), caprylic acid (C8) esters (less than 15%), and capric acid (C10) esters (less than 12%). GELUCIRE® 44/14 may also include free glycerol (typically less than about 1%). In a representative formulation, GELUCIRE® 44/14 includes lauric acid (C12) esters (about 47%), myristic acid (C14) esters (about 18%), palmitic acid (C16) esters (about 10%), stearic acid (C18) esters (about 11%), caprylic acid (C8) esters (about 8%), and capric acid (C10) esters (about 12%).

Palmitic acid (C16) (40-50%) and stearic acid (C18) (48-58%) are the predominant fatty acid components of the glycerides and polyethylene glycol esters in GELUCIRE® 50/13. GELUCIRE® 50/13 is known as PEG-32 glyceryl palmitostearate (Gattefosse), stearoyl macrogolglycerides EP, or stearoyl polyoxylglycerides USP/NF). GELUCIRE® 50/13 includes palmitic acid (C16) esters (40 to 50%), stearic acid (C18) esters (48 to 58%) (stearic and palmitic acid esters greater than about 90%), lauric acid (C12) esters (less than 5%), myristic acid (C14) esters (less than 5%), caprylic acid (C8) esters (less than 3%), and capric acid (C10) esters (less than 3%). GELUCIRE® 50/13 may also include free glycerol (typically less than about 1%). In a representative formulation, GELUCIRE® 50/13 includes palmitic acid (C16) esters (about 43%), stearic acid (C18) esters (about 54%) (stearic and palmitic acid esters about 97%), lauric acid (C12) esters (less than 1%), myristic acid (C14) esters (about 1%), caprylic acid (C8) esters (less than 1%), and capric acid (C10) esters (less than 1%) Stearic acid (C18) is the predominant fatty acid component of the glycerides and polyethylene glycol esters in GELUCIRE® 53/10. GELUCIRE® 53/10 is known as PEG-32 glyceryl stearate (Gattefosse). In one embodiment, the polyethylene oxide-containing fatty acid ester is a lauric acid ester, a palmitic acid ester, or a stearic acid ester (i.e., mono- and di-lauric acid esters of polyethylene glycol, mono- and di-palmitic acid esters of polyethylene glycol, mono- and di-stearic acid esters of polyethylene glycol). Mixtures of these esters can also be used.

For embodiments that include polyethylene oxide-containing fatty acid esters, the ratio of the fatty acid glycerol esters to polyethylene oxide-containing fatty acid esters is from about 20:80 to about 80:20 v/v. In one embodiment, the ratio of the fatty acid glycerol esters to polyethylene oxide-containing fatty acid esters is about 30:70 v/v. In one embodiment, the ratio of the fatty acid glycerol esters to polyethylene oxide-containing fatty acid esters is about 40:60 v/v. In one embodiment, the ratio of the fatty acid glycerol esters to polyethylene oxide-containing fatty acid esters is about 50:50 v/v. In one embodiment, the ratio of the fatty acid glycerol esters to polyethylene oxide-containing fatty acid esters is about 60:40 v/v. In one embodiment, the ratio of the fatty acid glycerol esters to polyethylene oxide-containing fatty acid esters is about 70:30 v/v.

As used herein, the term “polyethylene oxide-containing phospholipid” refers to a phospholipid that includes a polyethylene oxide group (i.e., polyethylene glycol group) covalently coupled to the phospholipid, typically through a carbamate or an ester bond. Phospholipids are derived from glycerol and can include a phosphate ester group and two fatty acid ester groups. Suitable fatty acids include saturated and unsaturated fatty acids having from eight (8) to twenty-two (22) carbons atoms (i.e., C8-C22 fatty acids). In certain embodiments, suitable fatty acids include saturated C12-C18 fatty acids. Representative polyethylene oxide-containing phospholipids include C8-C22 saturated fatty acid esters of a phosphatidyl ethanolamine polyethylene glycol salt. In certain embodiments, suitable fatty acids include saturated C12-C18 fatty acids. The molecular weight of the polyethylene oxide group of the polyethylene oxide-containing phospholipid can be varied to optimize the solubility of the therapeutic agent (e.g., the one or more proteasome inhibitors) in the formulation. Representative average molecular weights for the polyethylene oxide groups can be from about 200 to about 5000 (e.g., PEG 200 to PEG 5000).

In one embodiment, the polyethylene oxide-containing phospholipids are distearoyl phosphatidyl ethanolamine polyethylene glycol salts. Representative distearoylphosphatidyl ethanolamine polyethylene glycol salts include distearoylphosphatidyl ethanolamine polyethylene glycol 350 (DSPE-PEG-350) salts, distearoylphosphatidyl ethanolamine polyethylene glycol 550 (DSPE-PEG-550) salts, distearoylphosphatidyl ethanolamine polyethylene glycol 750 (DSPE-PEG-750) salts, distearoylphosphatidyl ethanolamine polyethylene glycol 1000 (DSPE-PEG-1000) salts, distearoylphosphatidyl ethanolamine polyethylene glycol 1500 (DSPE-PEG-1500) salts, and distearoylphosphatidyl ethanolamine polyethylene glycol 2000 (DSPE-PEG-2000) salts. Mixtures can also be used. For the distearoylphosphatidyl ethanolamine polyethylene glycol salts above, the number (e.g., 350, 550, 750, 1000, and 2000) designates the average molecular weight of the polyethylene oxide group. The abbreviations for these salts used herein is provided in parentheses above. Suitable distearoylphosphatidyl ethanolamine polyethylene glycol salts include ammonium and sodium salts.

The chemical structure of distearoylphosphatidyl ethanolamine polyethylene glycol 2000 (DSPE-PEG-2000) ammonium salt is comprised of a polyethylene oxide-containing phospholipid includes a phosphate ester group and two fatty acid ester (stearate) groups, and a polyethylene oxide group covalently coupled to the amino group of the phosphatidyl ethanolamine through a carbamate bond.

The polyethylene oxide-containing phospholipid affects the ability of the formulation to solubilize a therapeutic agent. In general, the greater the amount of polyethylene oxide-containing phospholipid, the greater the solubilizing capacity of the formulation for difficulty soluble therapeutic agents. The polyethylene oxide-containing phospholipid can be present in the formulation in an amount from about 1 mM to about 30 mM based on the volume of the formulation. In certain embodiments, the distearoylphosphatidyl ethanolamine polyethylene glycol salt is present in the formulation in an amount from 1 mM to about 30 mM based on the volume of the formulation. In one embodiment, the distearoylphosphatidyl ethanolamine polyethylene glycol salt is present in the formulation in about 15 mM based on the volume of the formulation.

In certain embodiments, the one or more proteasome inhibitors is provided to the subject in a solid dosage form (e.g., solid or semi-solid dosage forms) comprising bortezomib. In particular embodiments, the solid dosage form comprises an oral formulation disclosed herein. In some embodiments, the solid dosage form comprises bortezomib and at least one lipophilic component which are coated on a solid carrier. In other embodiments, the % w/w of bortezomib in the solid dosage form is greater than a % w/w of the at least one lipophilic component. In further embodiments, the % w/w of bortezomib is in the range of about 20% to about 30% of the total weight of the solid dosage form. In some embodiments, bortezomib is present in the solid dosage form in an amount in the range of from about 50 mg to about 200 mg. In other embodiments, bortezomib is present in amount of about 100 mg. In still other embodiments, wherein the bortezomib is present in amount of about 150 mg. In particular embodiments, the formulation is present in a hard shell capsule. In particular embodiments, the bortezomib formulation is provide orally. In certain embodiments, the solid dosage form in any of those shown in Tables 4-10.

In some embodiments, the at least one lipophilic component is selected from the group consisting of a polyethylene oxide-containing fatty acid ester, fatty acid glycerol ester, and a combination thereof. In some embodiments, the solid dose formulation comprises bortezomib, a polyethylene oxide-containing fatty acid ester, and fatty acid glycerol ester.

The solid dosage forms of the present disclosure can be prepared by any suitable method, including granulation of the therapeutic agent (e.g. the one or more proteasome inhibitors) with excipients (e.g. fillers, glidants, lubricants, etc. known in the art and described herein), extrusion of the therapeutic agent with excipients, direct compression of the therapeutic agent with excipients to form tablets, etc. In particular embodiments, the solid dosage forms the present disclosure can be prepared by coating the active agent, e.g. bortezomib on a solid carrier. The solid carrier can be any material upon which a drug-containing composition can be coated and which is suitable for human consumption. Any conventional coating process can be used. For example, the therapeutic agent, e.g. bortezomib can be dissolved or suspended in a suitable solvent (e.g., ethanol), together with an optional binder, or alternatively one or more of the lipophilic components described herein, and deposited on the solid carrier by methods known in the art, e.g. fluidized bed coating or pan coating methods. The solvent can be removed e.g. by drying, or in situ during the coating process (e.g., during fluidized bed coating), and/or in a subsequent drying step.

In some embodiments, the solid carrier may be an inert bead or an inert particle. In other embodiments, the solid carrier a non-pareil seed, an acidic buffer crystal, an alkaline buffer crystal, or an encapsulated buffer crystal. In some embodiments, the solid carrier may be a sugar sphere, cellulose sphere, lactose sphere, lactose-microcrystalline cellulose (MCC) sphere, mannitol-MCC sphere, or silicon dioxide sphere. In other embodiments, the solid carrier may be a saccharide, a sugar alcohol, or combinations thereof. Suitable saccharides include lactose, sucrose, maltose, and combinations thereof. Suitable sugar alcohols include mannitol, sorbitol, xylitol, maltitol, arabitol, ribitol, dulcitol, iditol, isomalt, lactitol, erythritol and combinations thereof. In embodiments, the solid carrier may be formed by combining any of the above with a filler. Examples of suitable fillers which may be used to form a solid carrier include lactose, microcrystalline cellulose, silicified microcrystalline cellulose, mannitol-microcrystalline cellulose and silicon dioxide. In other embodiments, the dosage form disclosed herein does not include a solid carrier. In other embodiments, the disclosure provides for a capsule comprising a solid dosage form described herein. Bortezomib oral dose formulations can be prepared using the formulations set out in any of Tables 4-8.

TABLE 4
Bortezomib Formulation 1

| Item | Ingredient | mg/unit |
|---|---|---|
| a | Bortezomib | 3.5 |
| b | Mannitol 160C | 150 |
| c | Tabulose 101 | 149 |
| d | Colloidal silicon dioxide | 10 |
| e | TPGS | 1 |
| f | Peceol | 10 |
| g | Gelucire 44/14 | 10 |
| h | Ethanol 100% (evaporated during the process) | — |
| i | Magnesium stearate | 5 |

Items a-h are internal phase components, and item i is the external phase component.

TABLE 5
Bortezomib Formulation 2

| Item | Ingredient | mg/unit |
|---|---|---|
| a | Bortezomib | 3.5 |
| b | Prosolv HD90 | 287 |
| c | Croscarmellose sodium | 22 |
| d | TPGS | 1 |
| e | Peceol | 10 |
| f | Gelucire 44/14 | 10 |
| g | Ethanol 100% (evaporated during the process) | — |
| h | Magnesium stearate | 5 |

Items a-g are internal phase components, and item h is the external phase component.

TABLE 6
Bortezomib Formulation 3

| Item | Ingredient | mg/unit |
|---|---|---|
| a | Bortezomib | 3.5 |
| b | Tabulose 101 | 287 |
| c | Plasdone K-29/32 | 22 |
| d | TPGS | 1 |
| e | Peceol | 10 |
| f | Gelucire 44/14 | 10 |
| g | Ethanol 100% (evaporated during the process) | — |
| h | Magnesium stearate | 5 |

Items a-g are internal phase components, and item h is the external phase component.

TABLE 7
Formulation 4

| Item | Ingredient | mg/unit |
|---|---|---|
| A | Bortezomib | 3.5 |
| B | Mannitol 160C | 150 |
| C | Tabulose 101 | 149 |
| D | Colloidal silicon dioxide | 10 |
| E | TPGS | 1 |
| F | Peceol | 10 |
| G | Gelucire 44/14 | 10 |
| H | Ethanol 100% (evaporated during the process) | — |
| I | Magnesium stearate | 5 |

Items a-h are internal phase components, and item i is the external phase component.

TABLE 8
Bortezomib Formulation 5

| Item | Ingredient | mg/unit |
|---|---|---|
| a | Bortezomib | 100 |
| b | Prosolv HD90 | 287 |
| c | Croscarmellose sodium | 22 |
| d | TPGS | 1 |
| e | Peceol | 10 |
| f | Gelucire 44/14 | 10 |
| g | Ethanol 100% (evaporated during the process) | — |
| h | Magnesium stearate | 5 |

Items a-g are internal phase components, and item h is the external phase component.

In Vitro Cell Testing

A number of different compounds were tested for efficacy in in vitro testing using the viral strain: 2019 Novel Coronavirus, Isolate USA-WA1/2020 (SARS-CoV-2) in human non-small-cell lung cancer cell line (Calu-3). The results are tabulated in Table 9 below.

Efficacy was tested in parallel in human non-small-cell lung cancer cell line (Calu-3 cells). Each test compound was tested individually. Technicians were blinded to the identification of the drug being tested. Each of the concentrations was evaluated in triplicate for efficacy.

Calu-3 lung cells were cultured in 96 well plates prior to the day of the assay. Cells demonstrated greater than 90% confluency at the start of the study. Each of the test compound concentrations was evaluated in triplicate.

Test article concentrations were tested in two different conditions:
1) Pre-treatment for 24±4 hours prior to virus inoculation followed by treatment immediately after removal of virus inoculum or
2) treatment only with test article added immediately following removal of virus inoculum.

Remdesivir was added immediately following removal of virus inoculum. For pre-treatment and treatment, wells will be overlaid with 0.2 mL DMEM2 (Dulbecco's Modified Eagle Media (DMEM) with 2% Fetal Bovine Serum (FBS) with test articles.

Following the 24±4 hour pre-treatment, cells were inoculated at a MOI of 0.001 TCID50/cell with SARS-CoV-2 and incubated for 60 to 90 minutes. Immediately following the 60 to 90 minute incubation, virus inoculum was removed, cells were washed and the appropriate wells overlaid with 0.2 mL DMEM2 (DMEM with 2% FBS with test or control articles) and incubated in a humidified chamber at 37° C.±2° C. in 5±2% CO₂. At 48±6 hours post inoculation, cells were fixed and evaluated for the presence of virus by immunostaining assay

The immunostaining assay modified the incubation time to 48 hours. A 24±4 hour pre-treatment of the cells is now included for selected test articles.

Immunostaining Assay: After 48±6 hours, cells were fixed with paraformaldehyde and stained using an anti-SARS-2 nucleoprotein monoclonal antibody (Sino Biological) followed by peroxidase-conjugated goat anti-mouse IgG (SeraCare). Wells were developed using TMB Substrate Solution and the reaction was stopped by acidification. The ELISA plate was read at 450 nm on a spectrophotometer by an ELISA plate reader.

For each well, the inhibition of virus was calculated as the percentage of reduction of the absorbance value in respect of the virus control by the following formula: percent inhibition=100−[(A450 of test article dilution−A450 of cell control)/(A450 of virus control−A450 of cell control)]×100. The EC50 was defined as the reciprocal dilution that caused 50% reduction of the absorbance value of the virus control (50% A450 reduction).

Bortezomib showed a significant reduction in the TCID50 titer, with the 50% effective concentration (EC50) of 7.8 nM.

TABLE 9
Summary of Preliminary Results in Lung Cells

| Drug | EC50 | | EC100 | AI Probability Score |
|---|---|---|---|---|
| Bortezomib | 6.9 | nM* | Achieved | (T = 0.06) |
| Remdesivir | 252 | nM* | Achieved | N/A |

*Significant Viral Activity

Second Testing Series

Efficacy was tested in parallel in African green monkey kidney (Vero E6) cells. Each test compound was tested individually. Technicians were blinded to the identification of the drug being tested. Each of the concentrations was evaluated in triplicate for efficacy. Vero E6 cells were cultured in 96 well plates prior to the day of the assay. Cells were greater than 90% confluency at the start of the study. Each of the test article concentrations was evaluated in triplicate.

Test article concentrations was tested in two different conditions: 1) Pre-treatment for 24±4 hours prior to virus inoculation followed by treatment immediately after removal of virus inoculum or 2) treatment only with test article added immediately following removal of virus inoculum. Remdesivir was added immediately following removal of virus inoculum. For pre-treatment and treatment, wells were overlaid with 0.2 mL DMEM2 (Dulbecco's Modified Eagle Media (DMEM) with 2% Fetal Bovine Serum (FBS) with test compounds at various concentrations. Following the 24±4 hour pre-treatment, cells were inoculated at a MOI of 0.001 TCID50/cell with SARS-CoV-2 and incubated for 60 to 90 minutes.

Immediately following the 60 to 90 minute incubation, virus inoculum was removed, cells were washed and the appropriate wells were overlaid with 0.2 mL DMEM2 (DMEM with 2% FBS with test or control articles) and incubated in a humidified chamber at 37° C.±2° C. in 5±2% CO2. At 48±6 hours post inoculation, cells were fixed and evaluated for the presence of virus by immunostaining assay. The immunostaining assay utilized modified the incubation time to 48 hours. A 24±4 hour pre-treatment of the cells was included for selected test articles.

TABLE 10
Efficacy in African green monkey kidney (Vero E6) cells
infected with Virus (Viral Strain used: 2019 Novel
Coronavirus, Isolate USA-WA1/2020 (SARS-CoV-2)Code

| Drug | EC50 | | EC₁₀₀ | comment |
|---|---|---|---|---|
| Bortezomib | 9.92 | nM | Achieved | |
| Remdesevir | 1.15 | um | Achieved | Positive control |

**Activity based on concentrations used to treat tapeworms(3.10-9to 3.10-5M) and systemic fungal infections(1000-2000 nM).

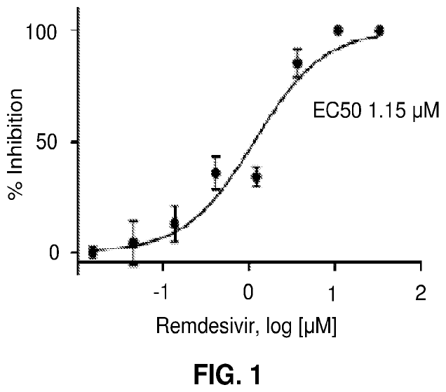

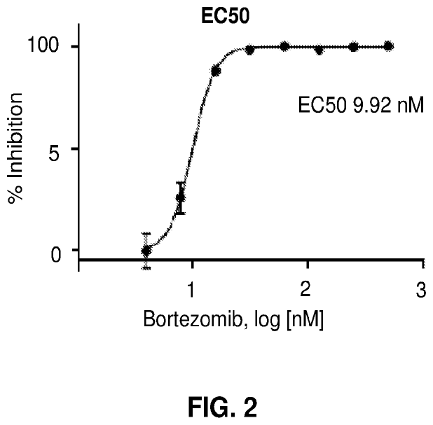

The above results of Table 10 confirm the results obtained in the first testing series and confirm the versatility of Bortezomib as a potent inhibitor of COVID-19 as the tests were carried out on different strains of COVID-19. The above results also indicate that in this test, bortezomib was clearly superior to Remdesevir in terms of EC50 as also evidenced in FIGS. 1 and 2.

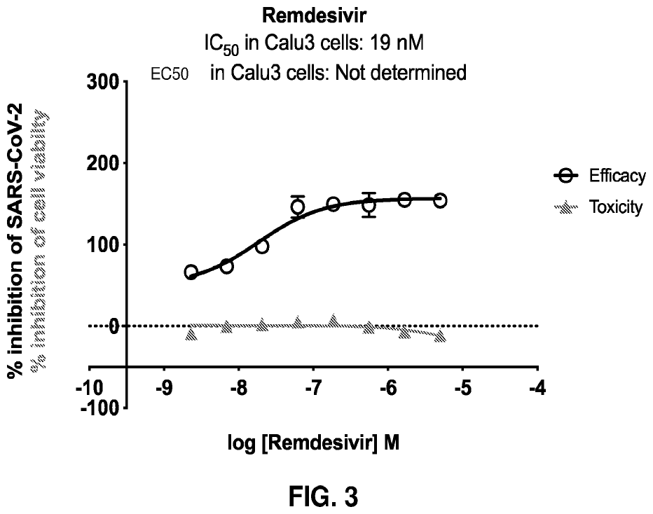

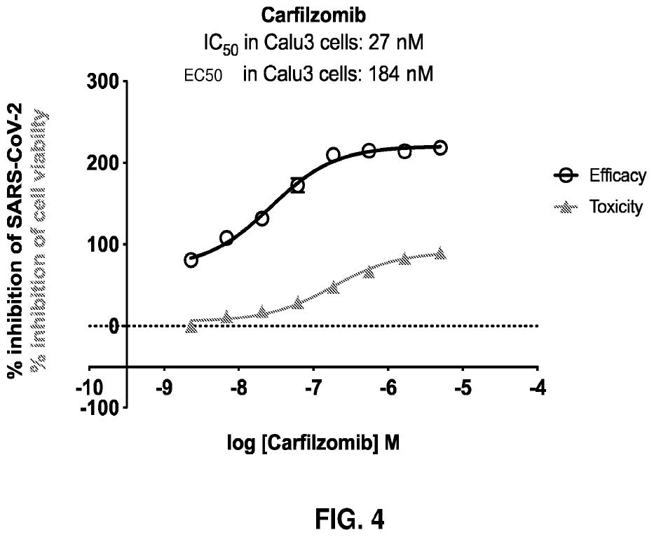

FIG. 3 and FIG. 4 show the IC50 and EC50 values determined using the methods described above in Calu-3 cells that were exposed to SARS-CoV-2 virus and treated with remdesivir (FIG. 3) or carfilzomib (FIG. 4). Without being bound by any particular theory, the data shown in FIG. 4 supports the use of carfilzomib as a therapy for ameliorating and/or inhibiting some or substantially all of the risks, symptoms and development of severe disease in a subject infected the SARS-CoV-2 virus.

1. A method for preventing a SARS-CoV-2 infection in a subject comprising: administering an oral formulation comprising one or more proteasome inhibitors to the subject.

2. A method for treating a SARS-Co V-2 infection in a subject comprising: administering an oral formulation comprising one or more proteasome inhibitors to the subject.

3. A method for preventing replication of SARS-CoV-2 virus in a subject comprising: administering an oral formulation comprising one or more proteasome inhibitors to the subject.

4. The use method according to claim 1 wherein the one or more proteasome inhibitors comprises bortezomib.

5. The use method according to claim 1 wherein the one or more proteasome inhibitors comprises Carfilzomib.

6. The use method according to claim 1 wherein the one or more proteasome inhibitors comprises Ixazomib.

7. The method according to claim 1 where the one or more proteasome inhibitors is selected from bortezomib, Carfilzomib, Ixazomib and a combination thereof.

8. The method according to claim 2, where the one or more proteasome inhibitors is selected from bortezomib, Carfilzomib, Ixazomib and a combination thereof.

9. The method according to claim 2, wherein the one or more proteasome inhibitors comprises bortezomib.

10. The method according to claim 2, wherein the one or more proteasome inhibitors comprises Carfilzomib.

11. The method according to claim 2, wherein the one or more proteasome inhibitors comprises Ixazomib.

12. The method according to claim 3, where the one or more proteasome inhibitors is selected from bortezomib, Carfilzomib, Ixazomib and a combination thereof.

13. The method according to claim 3, wherein the one or more proteasome inhibitors comprises bortezomib.

14. The method according to claim 3, wherein the one or more proteasome inhibitors comprises Carfilzomib.

15. The method according to claim 3, wherein the one or more proteasome inhibitors comprises Ixazomib.
